(19) United States
(12) Patent Application Publication
(10) Pub. No.: US 2011/0166511 A1
(43) Pub. Date: Jul. 7, 2011

(54) DROP CONTROLLING AND COUNTING - VALVE ON KEY

(75) Inventors: Pierre Sharvit, Emeq Hefer (IL); Jacob Nusbacher, Ra'anana (IL); Gershon Goldenberg, Carcur (IL); Michal Devir, Kfar Vitkin (IL); Youval Katzman, Zichron Yaakov (IL)

(73) Assignee: PRO-IV LTD., KFAR VITKIN (IL)

(21) Appl. No.: 12/935,329

(22) PCT Filed: Apr. 1, 2009
(86) PCT No.: PCT/IB09/51379

§ 371 Date: Mar. 25, 2011

Related U.S. Application Data
(60) Provisional application No. 61/064,902, filed on Apr. 2, 2008.

(51) Int. Cl. A61M 5/00

(52) U.S. Cl. 604/65

(57) ABSTRACT

A drop controlling and counting valve on key system, and a method for ensuring authentication and for controlling the rate of flow of medications, in liquid state drops, under control of a hand-held computer serving as an authentication unit, the hand-held computer containing characteristics of the medication fluid and details of the patient, for calculating a correlation value between the details and the characteristics and having a control unit serving as a key for opening and controlling passage of the medication dripping through a smart valve.

### FIELD OF THE INVENTION

The present invention relates to means and a method designated to prevent medical errors when injecting IV fluids and medications into humans and animals, and, in particular to ensure authentication of medications infused in IV bags.

### BACKGROUND OF THE INVENTION

An apparatus, system and method for administration of a substance is described in the International Application PCT/IL/2005/001118 of Sharvit et al., International Publication Number WO 2006/046242, which is incorporated by reference for all purposes as if fully set forth herein.

WO 2006/046242 discloses an infusion control valve adapted to be actuated by a valve actuator, an infusion valve actuator adapted to actuate an infusion control valve upon being triggered by an authentication unit and a method for the administration of a substance.

The method according to WO 2006/046242 also uses a hand-held (HHD) computer and a smart (electronic) key.

Means and a method of prevention of error and ensuring authentication of medications infused in IV bags and syringes, and other authentication, such as the verification of movement of fluids in all directions from bags to vials, bags to syringes, and syringes to vials, is described in the U.S. provisional patent application No. 61/006,578 of Sharvit et al., which is incorporated by reference for all purposes as if fully set forth herein.

U.S. 61/006,578 discloses a drug port valve which has two working modes, a closed mode which completely prevents the passage of fluid, and an open mode which requires authentication and which enables the passage of fluid.

There is a need for a means and a method designated to prevent medical errors when injecting IV fluids and medications into humans and animals, and, in particular to ensure authentication of medications infused in IV bags, which enable controlling and monitoring the output of IV fluid passing through such a valve.

### SUMMARY OF THE INVENTION

The present invention relates to system, means and a method of use, designated to prevent medical errors when injecting IV fluids and medications into humans and animals, and, in particular to ensure authentication of medications infused in IV bags, which enable control and monitoring the output of IV fluid.

The flow through the means is at a dripping rate, as is common in fluid IV's, and the system, according to the present invention, enables closed circuit monitoring of the output, namely the dripping rate, while the mass of the drops is known and enables selection of desired output parameters, such as the number of drops per time unit and the beginning and end times of the flow, all under the condition of authentication.

These system, means and method are according to the present invention, some of whose inventors are also inventors of WO 2006/046242, and U.S. 61/006,578 and are designated to add further performance to the family of system, means, and method of the prior invention.

According to some embodiments of the present invention there is provided a drop controlling and counting valve on key system for ensuring authentication and for controlling the rate of flow of medications, in liquid state drops, under control of an authentication unit, the authentication unit containing characteristics of the medication fluid and details of the patient, for calculating a correlation value between the details and the characteristics, the drop controlling and counting valve on key system including: (A) a smart valve including: (i) an immovable assembly including: (a) a smart valve to control unit connector; and (B) a control unit including: (i) a control unit to smart valve connector, wherein the smart valve to control unit connector and the control unit to smart valve connector are compatible; and (ii) a control unit wireless communication subsystem.

According to still further features in the described embodiments the drop control and controlling valve on key system further includes: (C) a hand-held computer including: (i) a hand-held computer wireless communication subsystem, wherein the control unit wireless communication subsystem and the hand-held computer wireless communication subsystem are compatible.

According to still further features in the described embodiments the immovable assembly further includes: (b) a lock pin, having no movement capability relative to the immovable assembly; (c) a dripping chamber positioned at a lower section of the immovable assembly at times of a normal operation; (d) a lower connector attached to the dripping chamber; (e) a transmitter light guide disposed between the dripping chamber and the smart valve to control unit connector; and (f) a receiver light guide disposed between the dripping chamber and the smart valve to control unit connector.

According to still further features in the described embodiments the smart valve further includes: (ii) a moveable assembly, wherein the moveable assembly has a limited movement capability within the immovable assembly, and wherein the immovable assembly includes: (a) a spike having a shape and dimensions suitable for insertion in an IV bag first port.

According to still further features in the described embodiments the smart valve further includes: (iii) an internal tubule disposed between the spike and the lower connector.

According to still further features in the described embodiments the moveable assembly further includes (b) a lock having angular movement capability, wherein the lock does not block flow of fluid within the internal tubule during times of storage; (c) a lock hook for locking the lock in a position pressing on the internal tubule; and (d) a drop controller means for controlling the rate of fluid dripping through the internal tubule.

According to still further features in the described embodiments the control unit further includes: (iii) an optical transmitter, wherein when the control unit is engaged to the smart valve, the optical transmitter is positioned opposite the transmitter light guide; (iv) an optical receiver, wherein when the control unit is engaged to the smart valve, the optical transmitter is positioned opposite the receiver light guide; and a control unit locker having angular movement capability, and wherein when the control unit is connected to the smart valve, the control unit locker can prevent disengagement of the control unit from the smart valve.

According to still further features in the described embodiments the control unit further includes: (vi) a locking shaft having rotational movement capability; (vii) a combining ligule disposed as part of the locking shaft, wherein the combining ligule has a shape and dimensions suitable for engagement with the drop controller means; and (viii) a cam disposed as part of the locking shaft, wherein the cam has a shape and dimensions suitable for moving the control unit lock in order to enable disengagement of the control unit from the smart valve.

According to still further features in the described embodiments the control unit further includes: (ix) a step motor, the step motor having a step motor shaft; (x) a first cogwheel disposed at the step motor shaft; and (xi) a second cogwheel disposed at the locking shaft, wherein the first cogwheel and the second cogwheel constitute a control transmission.

According to still further features in the described embodiments the control unit further includes: (xii) a microcontroller capable of operating the step motor; and (xiii) a power source, for supplying power to the step motor and to the micro-computer.

According to still further features in the described embodiments the control unit further includes: (iii) an optical transmitter, wherein when the control unit is engaged to the smart valve, the optical transmitter is positioned opposite the transmitter light guide; (iv) an optical receiver, wherein when the control unit is engaged to the smart valve, the optical transmitter is positioned opposite the receiver light guide; a control unit lock having angular movement capability, and wherein when the control unit is connected to the smart valve, the control unit lock can prevent disengagement of the control unit from the smart valve; (vi) a locking shaft having rotational movement capability; (vii) a combining ligule disposed as part of the locking shaft, wherein the combining ligule has a shape and dimensions suitable for engagement with the drop controller means; (viii) a cam disposed as part of the locking shaft, wherein the cam has a shape and dimensions suitable for moving the control unit lock in order to enable disengagement of the control unit from the smart valve; (ix) a step motor, the step motor having a step motor shaft; (x) a first cogwheel disposed at the step motor shaft; (xi) a second cogwheel disposed at the locking shaft, wherein the first cogwheel and the second cogwheel constitute a control transmission; (xii) a microcontroller capable of operating the step motor; and (xiii) a power source, for supplying power to the step motor and to the micro-computer.

According to some embodiments of the present invention there is provided a method for controlling the rate of flow of medications, in liquid state drops, infused in IV bags, the method including the stages of: (A) providing a drop controlling and counting valve on key system, the drop controlling and counting valve on key system including: (i) a first smart valve having a spike; (ii) a control unit; and (iii) a hand-held computer; (B) inserting the spike in an IV bag port, wherein the insertion causes a state of prevention of fluid flow from the IV bag through the first smart valve; (C) connecting the control unit to the first smart valve; (D) scanning a vial barcode sticker and a wristband patient barcode by the hand-held computer, and assessing an authentication; (E) opening a pass which enables flow of fluid through the first smart valve; and (F) measuring the flow rate of fluid, by counting fluid drops passing through the first smart valve, over a given period of time, wherein the average mass of a drop is known.

According to still further features in the described embodiments the method for ensuring authentication and for controlling the rate of flow of medications, in liquid state drops, infused in IV bags further including the stages of: (G) calculating an amount of fluid mass passing through the first smart valve; and (H) preventing flow of fluid through the first smart valve, after finding that a fluid mass of a predetermined amount passed through the first smart valve.

According to still further features in the described embodiments the method for ensuring authentication and for controlling the rate of flow of medications, in liquid state drops, infused in IV bags further including the stages of: (I) disconnecting the control unit from the first smart valve; and (J) extracting the spike from the IV bag port.

According to still further features in the described embodiments the method for ensuring authentication and for controlling the rate of flow of medications, in liquid state drops, infused in IV bags further including the stages of: (K) destroying the first smart valve.

According to still further features in the described embodiments the method for ensuring authentication and for controlling the rate of flow of medications, in liquid state drops, infused in IV bags further including the stages of: (L) inserting a spike of a second smart valve in an IV bag port, wherein the insertion causes a state of prevention of fluid flow from the IV bag through the second smart valve; and (M) connecting the control unit to the second smart valve.

Additional objects and advantages of the invention will be set forth in part in the description which follows and, in part, will be obvious from the description, or may be learned by practice of the invention.

### BRIEF DESCRIPTION OF THE DRAWINGS

The invention is herein described, by way of example only, with reference to the accompanying drawings, wherein:

FIG. 1 is a schematic perspective view illustration of an exemplary embodiment of the three main assemblies of a drop controlling and counting valve on key system, according to the present invention.

FIG. 2 is a schematic perspective view illustration of an exemplary embodiment of an open control unit, without part of the external casing and additional parts, according to the present invention.

FIG. 3 is a schematic perspective view illustration of an exemplary embodiment of an open smart valve, according to the present invention.

FIG. 4 is a schematic front view illustration of an exemplary embodiment of the smart valve, according to the present invention, upon which the section plane a-a is marked.

FIG. 5 is a cross sectional view a-a schematic illustration of an exemplary, illustrative embodiment of the smart valve, prior to activation according to the present invention.

FIG. 6 is a schematic side view illustration of an exemplary embodiment of the control unit, according to the present invention.

FIG. 7 is a schematic perspective view illustration of an exemplary embodiment of a smart valve, according to the present invention, connected to infusion tubule about to be connected to IV bag, according to the present invention.

FIG. 8 is a schematic side view illustration of an exemplary embodiment of a smart valve, showing its components in a state in which flow is impossible, according to the present invention.

FIG. 9 is a schematic side view illustration of an exemplary embodiment of a smart valve, showing the state of its components after locking, according to the present invention.

FIG. 10 is a schematic side view illustration of an exemplary embodiment of a smart valve, which is connected to IV bag prior to connection to a control unit, according to the present invention.

FIG. 11 is a schematic side view illustration of an exemplary embodiment of a smart valve, which is connected to a control unit, according to the present invention.

FIG. 12 is a schematic side view illustration of an exemplary embodiment of a smart valve, which is connected to a control unit, according to the present invention.

FIG. 13 is a schematic perspective view illustration of an exemplary embodiment of a smart valve, integrated with a control unit and connected between an IV bag and an infusion tubule, according to the present invention.

FIG. 14 is a schematic perspective view illustration of an exemplary embodiment of a smart valve, integrated with a control unit and connected between an IV bag and an infusion tubule, according to the present invention.

FIG. 15 is a schematic perspective view illustration of an exemplary embodiment of a smart valve, integrated with a control unit and connected between an IV bag and an infusion tubule, according to the present invention.

FIG. 16 is a schematic side view illustration of an exemplary embodiment of a smart valve, which is connected to a control unit, according to the present invention.

FIG. 17 is a schematic perspective view illustration of an exemplary embodiment of a smart valve, integrated with a control unit and connected between an IV bag and an infusion tubule, according to the present invention, during adjustment of the control unit.

FIG. 18 is a schematic side view illustration of an exemplary embodiment of a smart valve, connected to a control unit, according to the present invention.

FIG. 19 is a schematic side view illustration of an exemplary embodiment of a smart valve, connected to a control unit, according to the present invention.

FIG. 20 is a schematic side view illustration of an exemplary embodiment of a smart valve, connected between an IV bag and an infusion tubule, according to the present invention, in the stage following disconnection from the control unit.

### DETAILED DESCRIPTION OF EMBODIMENTS

The present invention is of drop controlling and counting valve on key system, means and a method of use, designated to prevent medical errors when injecting IV fluids and medications into humans and animals, and, in particular to ensure authentication of medications infused in IV bags, which enable control and monitoring the output of IV fluid.

The flow, which is in the form of dripping, is through a valve and is controlled by a closed loop controlling sub-system, which can also provide a secure constant rate (according the physician protocol setup), namely, other than mass control it can also control a constant rate. An additional feature of the controlling sub-system is the ability for real-time reporting of every situation to the HHD by means of wireless communication, so that the HHD is updated from all units constantly during the procedure. The control can also include control of the time of beginning and end of dripping.

Even though in the embodiments described in the present patent application, the drop controlling and counting valve on key system includes one smart valve, one control unit, and one hand-held computer, there may be other embodiments in which one hand-held computer has wireless communication with more than one control unit.

The principles and operation of a drop controlling and counting valve on key system 1000 according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

The following list is a legend of the numbering of the application illustrations:
10 infusion bag barcode sticker
17 IV bag
18 IV bag first port
19 IV bag second port
20 infusion tubule
21 patient barcode
   30 fluid drops
   40 IR radiation
   41 wireless communication
   100 smart valve
   101 patient barcode
   102 vial barcode sticker
   103 dripping chamber
   104 smart valve to infusion tubule connector
   105 spike
   106 smart valve to control unit connector
   107 moveable assembly
   108 immovable assembly
   109 drop controller means
   110 transmitter light guide
   111 receiver light guide
   112 internal tubule
   113 lower connector
   114 lock hook
   115 lock
   116 lock pin
   117 pressure zone
   118 drop controller means plane
   119 locking wall
   120 transmitted light ray
   121 reflected light ray
   122 integral screw
   200 control unit
   201 external casing
   202 display
   203 keyboard
   204 control unit to smart valve connector
   205 switch
   206 step motor
   207 control transmission
   208 microcontroller
   209 power source
   210 optical transmitter
   211 optical receiver
   212 control unit lock
   213 first cogwheel
   214 second cogwheel
   215 step motor shaft
   216 locking shaft
   217 spring
   218 cam
   219 combining ligule
   220 control unit wireless communication subsystem
   300 hand-held computer
   301 LCD screen
   302 keypad
   303 IR radiation
   304 hand-held computer wireless communication subsystem
   1000 drop controlling and counting valve on key system

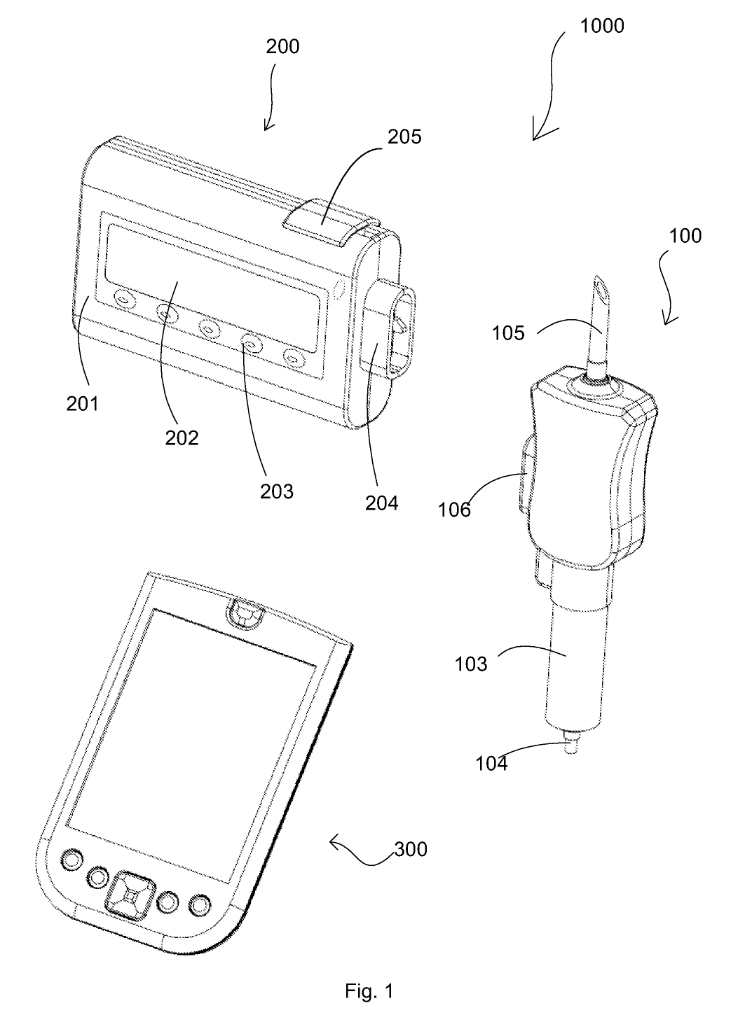

Referring now to the drawings, FIG. 1 is a schematic perspective view illustration of an exemplary embodiment of the three main assemblies of a drop controlling and counting valve on key system 1000, according to the present invention. The three main assemblies are: a smart valve 100 designated for one-time use, a control unit 200 designated for repeated use, and a hand-held (HHD) computer 300 which is also for repeated use.

The control unit 200 is suitable for connection to the smart valve 100 and for its activation. The hand-held (HHD) computer 300 enables the activation of the control unit 200 through wireless communication, following the connection and calibration of the control unit 200 and receiving a suitable authentication result from examination of the vial barcode sticker and the wristband patient barcode (21).

The smart valve 100 has spike 105 assembled to its upper part, and dripping chamber 103 assembled to its lower part. Dripping chamber 103 is a transparent cylinder which serves as a container for formation of the drops, and its lower end has a smart valve to infusion tubule connector 104.

The smart valve 100 also includes a smart valve to infusion tubule connector 104.

The control unit 200 also includes external casing 201 which is composed of a suitable material, such as plastic for example, and is integrated with a display 202 for displaying work data, as well as a keyboard 203 for entering data and a switch 205, which is a slider with two modes, connection and disconnection of the control unit 200 to and from the smart valve 100 by means of control unit to smart valve connector 204.

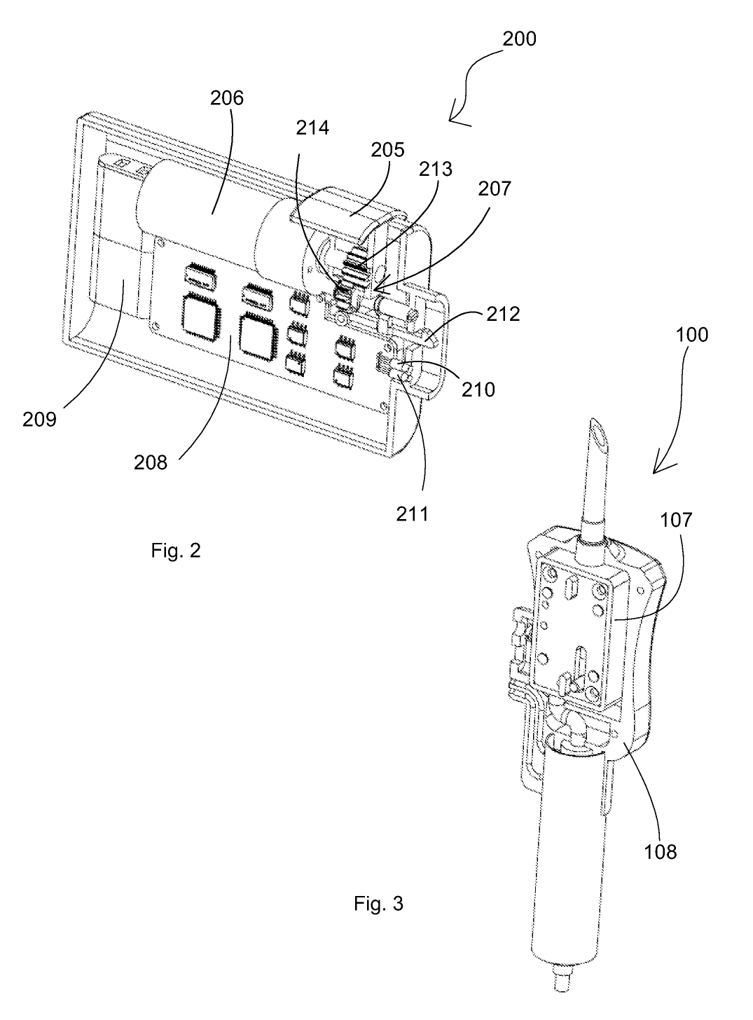

FIG. 2 is a schematic perspective view illustration of an exemplary embodiment of an open control unit 200, without part of the external casing 201 and additional parts, according to the present invention.

A motor, which can also be an electric step motor 206, fed from a power source 209, which can also be a chargeable electric battery, drives control transmission 207, which includes a first cogwheel 213 and a second cogwheel 214, and which controls (monitors) the dripping rate of the fluid drops flowing through the smart valve (100).

The control unit 200 also includes an optical transmitter 210, optical receiver 211, and microcontroller 208.

FIG. 3 is a schematic perspective view illustration of an exemplary embodiment of an open smart valve 100, according to the present invention.

The smart valve 100 includes two assemblies, an immovable assembly 108, and a moveable assembly 107, which moves when activated within the immovable assembly 108.

The terms moveable and immovable are used in reference to relative movement of these assemblies with regard to each other, and are in no way limiting their movement with regard to the external environment.

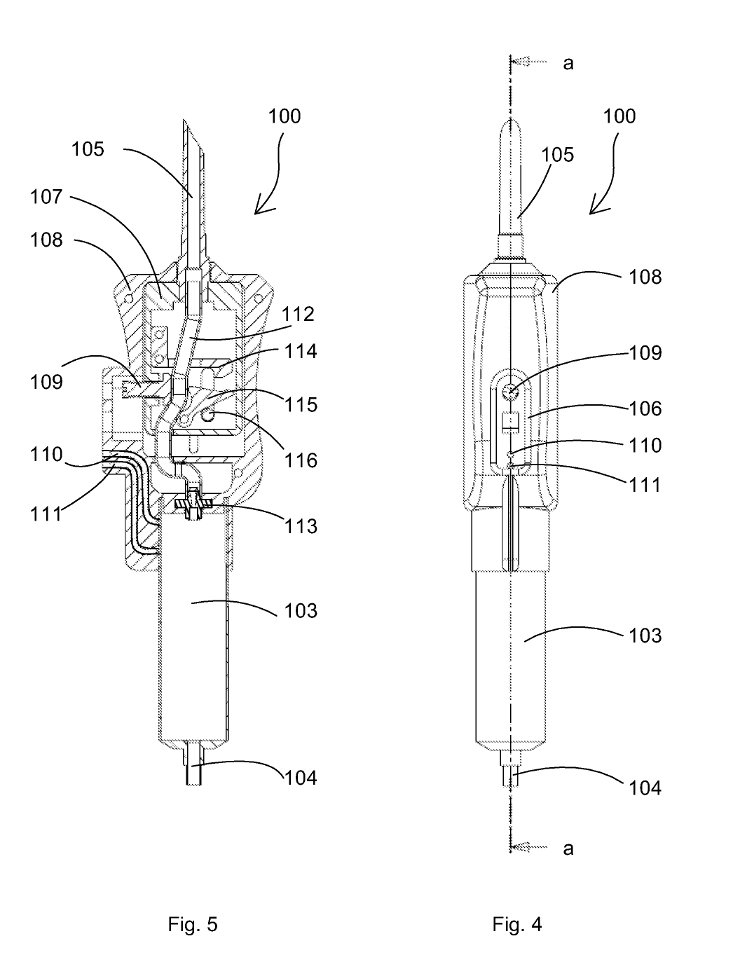

FIG. 4 is a schematic front view illustration of an exemplary embodiment of the smart valve 100, according to the present invention, upon which the section plane a-a is marked. The smart valve to control unit connector 106 also includes a drop controller 109, and two light guides, the transmitter light guide 110, and the receiver light guide 111.

FIG. 5 is a cross sectional view a-a schematic illustration of an exemplary, illustrative embodiment of the smart valve 100, prior to activation according to the present invention.

An internal tubule 112 goes through the moveable assembly 107 and is connected to lower connector 113. Drops can pass through the internal tubule 112 when there is flow of fluid into the dripping chamber 103. In this state, the lock hook 114 is in open mode when the lock 115 is in its lower position: likewise the lock pin 116, activates the lock by moving the movable assembly 107, movable assembly 107 is in the upper position.

The illustration shows the two light guides, the transmitter light guide 110, and the receiver light guide 111, serving for conduction of the light from the optical transmitter 210, and to the optical receiver 211 through the dripping chamber 103. In this state, the drop controller means 109 is in a fully closed mode.

There is still no flow through the internal tubule 112 because there has been no connection to any container of fluid.

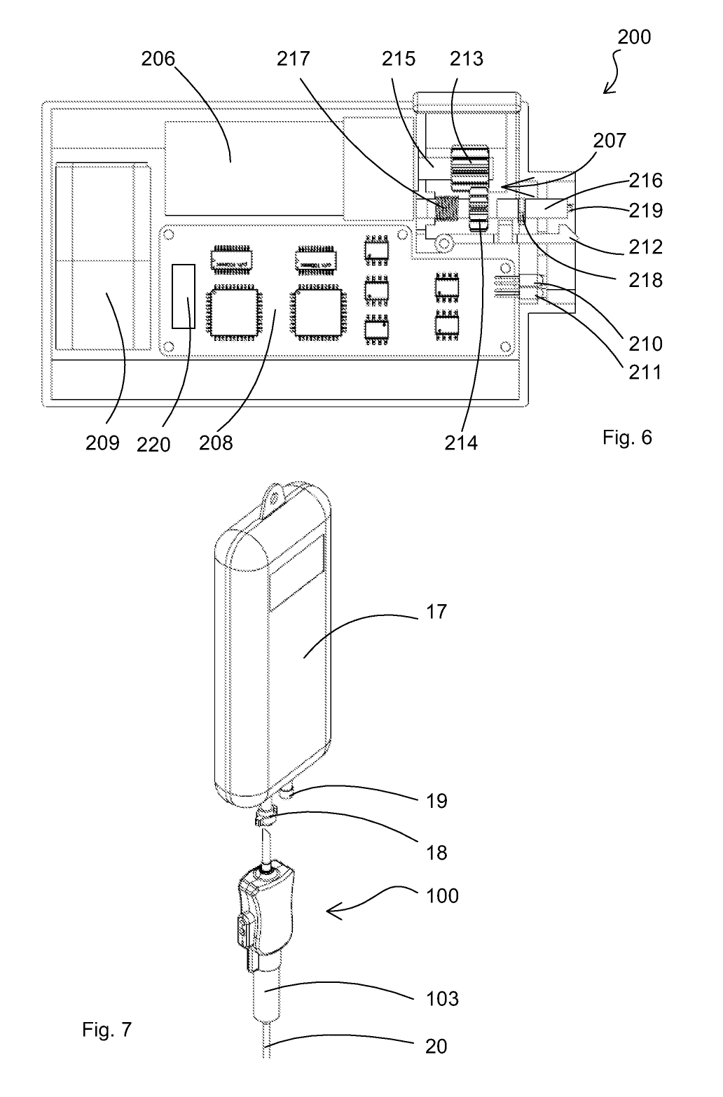

FIG. 6 is a schematic side view illustration of an exemplary embodiment of the control unit 200, according to the present invention.

The control unit 200 is activated by microcontroller 208 which is electrically connected to step motor 206, which activates the control transmission 207.

Step motor 206 has a step motor shaft 215, upon which a first cogwheel 213 is assembled and engaged with a second cogwheel 214, which is assembled to the locking shaft 216.

The locking shaft 216 is regularly engaged by spring 217.

The locking shaft 216 also includes a cam 218 serving to open the control unit lock 212. At the end of the locking shaft 216 is combining ligule 219, which is designated for controlling the dripping rate by opening and closing the drop controller means (109) which is disposed within smart valve (100).

The optical transmitter 210 also includes a light source such as LED, and the optical receiver 211 also includes a light-sensitive sensor.

FIG. 7 is a schematic perspective view illustration of an exemplary embodiment of a smart valve 100, according to the present invention, connected to infusion tubule 20 about to be connected to IV bag 17, according to the present invention. The connection is done by inserting spike 105 into the IV bag 17 through the IV bag first port 18.

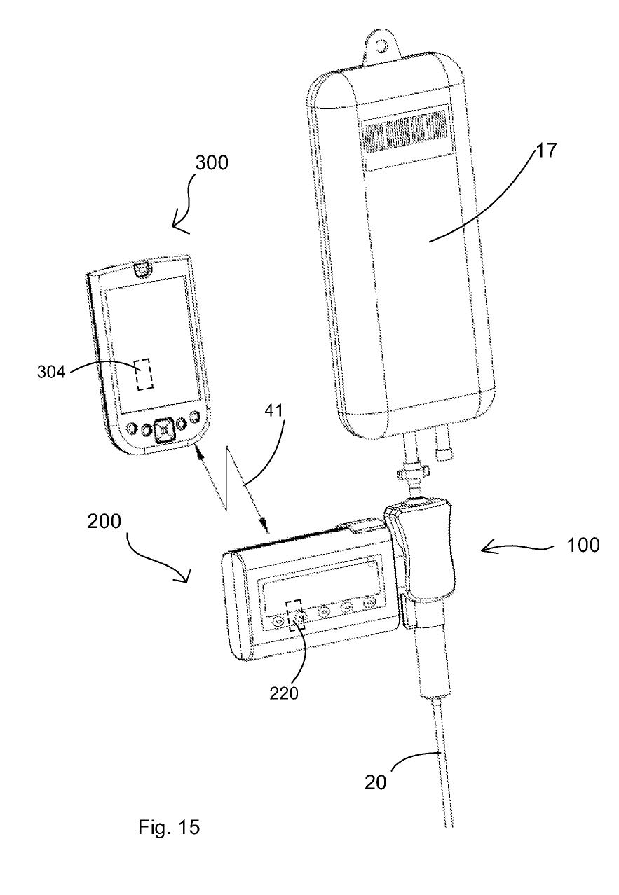

The illustration also shows a control unit wireless communication subsystem 220 which can be a little chip on a board of the microcontroller 208, and whose role will be explained in the description of FIG. 15.

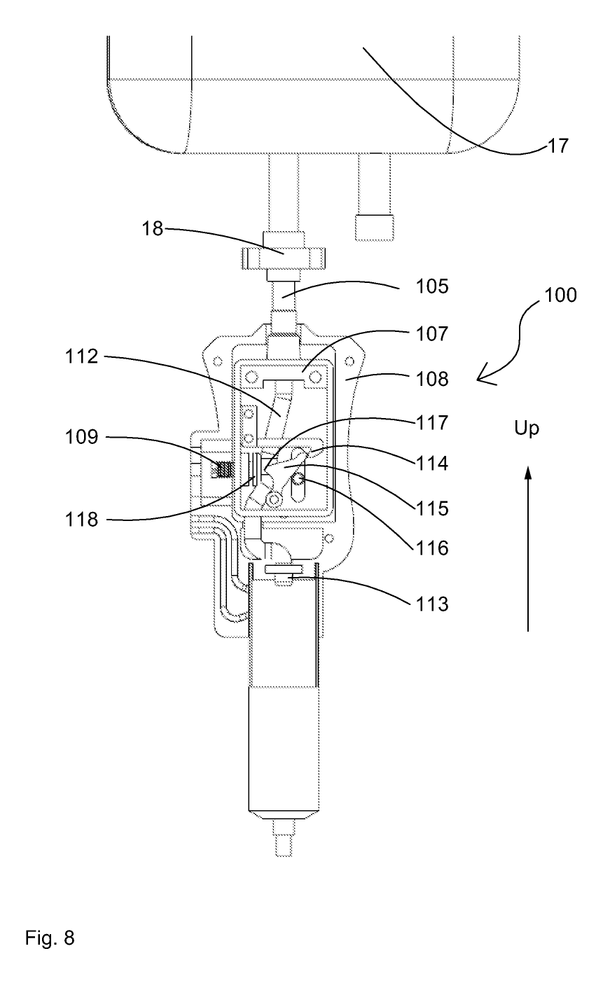

FIG. 8 is a schematic side view illustration of an exemplary embodiment of a smart valve 100, showing its components in a state in which flow is impossible, according to the present invention. The connection of the smart valve 100 to the IV bag 17, as described for the previous illustration, creates movement in the direction of the arrow up as shown in the illustration, which indicates movement of the moveable assembly 107 relative to the immovable assembly 108, and therefore the lock pin 116, which is part of the immovable assembly 108, in motion pushes the lock 115 towards the lock hook 114. The lock hook 114 enables lock 115 to pass it, but does not enable its return. In this state, the internal tubule 112 is completely pressed in pressure zone 117 so that no fluid can flow through pressure zone 117.

The drop controller means plane 118, which is at the end of the drop controller means 109, is fully closed. Namely, as shown in this illustration, the smart valve 100 is closed, and there is no dripping or continuous flow through the internal tubule 112.

The need for two modes of the lock hook 114 is a result of the requirement that during prolonged storage no force will be applied to the internal tubule 112, so that it is not damaged.

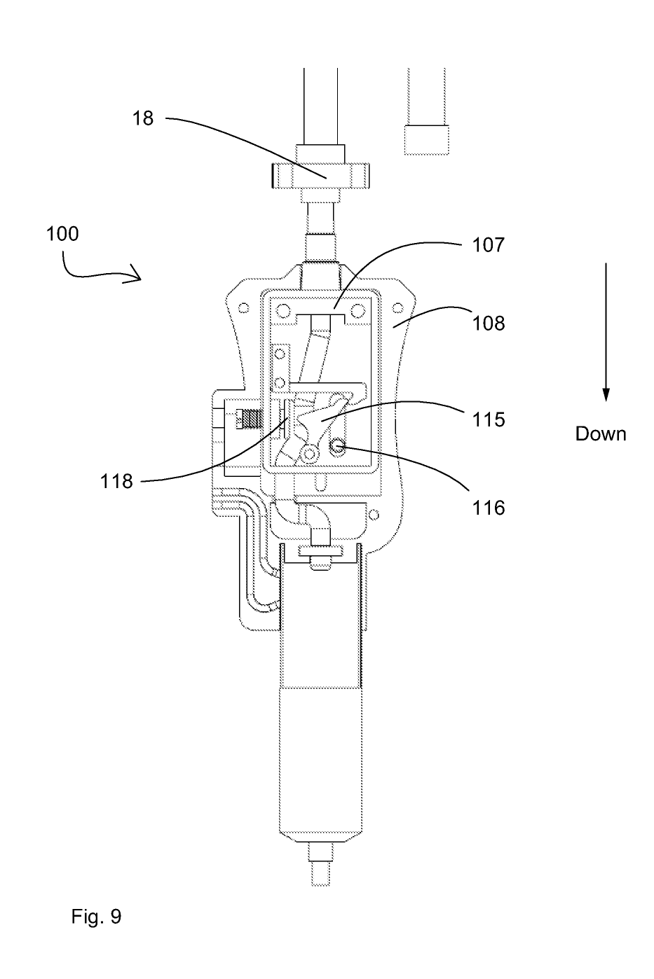

FIG. 9 is a schematic side view illustration of an exemplary embodiment of a smart valve 100, showing the state of its components after locking, according to the present invention. While the moveable assembly 107 remains attached to the IV bag first port 18 when the immovable assembly 108 moves back to its original position, down, as shown by the arrow in the illustration, the lock 115 remains closed, the lock pin 116 also returns to its original state, as shown in the illustration, and the drop controller means plane 118 also remains closed.

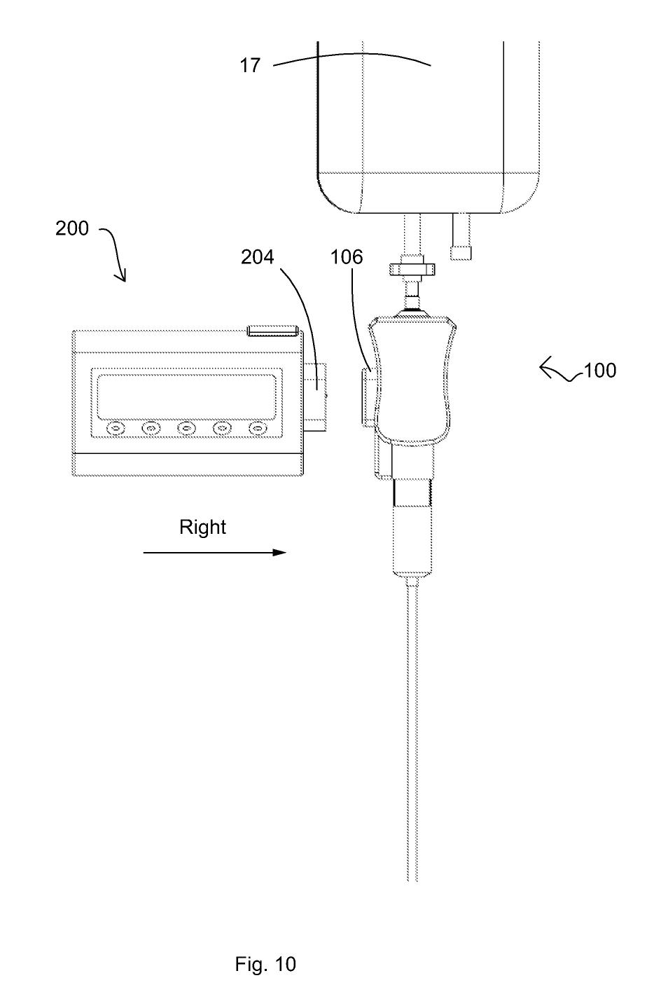

FIG. 10 is a schematic side view illustration of an exemplary embodiment of a smart valve 100 which is connected to IV bag 17 prior to connection to a control unit 200, according to the present invention. The connection of the control unit 200 to the smart valve 100, is by engaging the control unit to smart valve connector 204 with the smart valve to control unit connector 106 when moving the control unit 200 right, as shown by the arrow in the illustration.

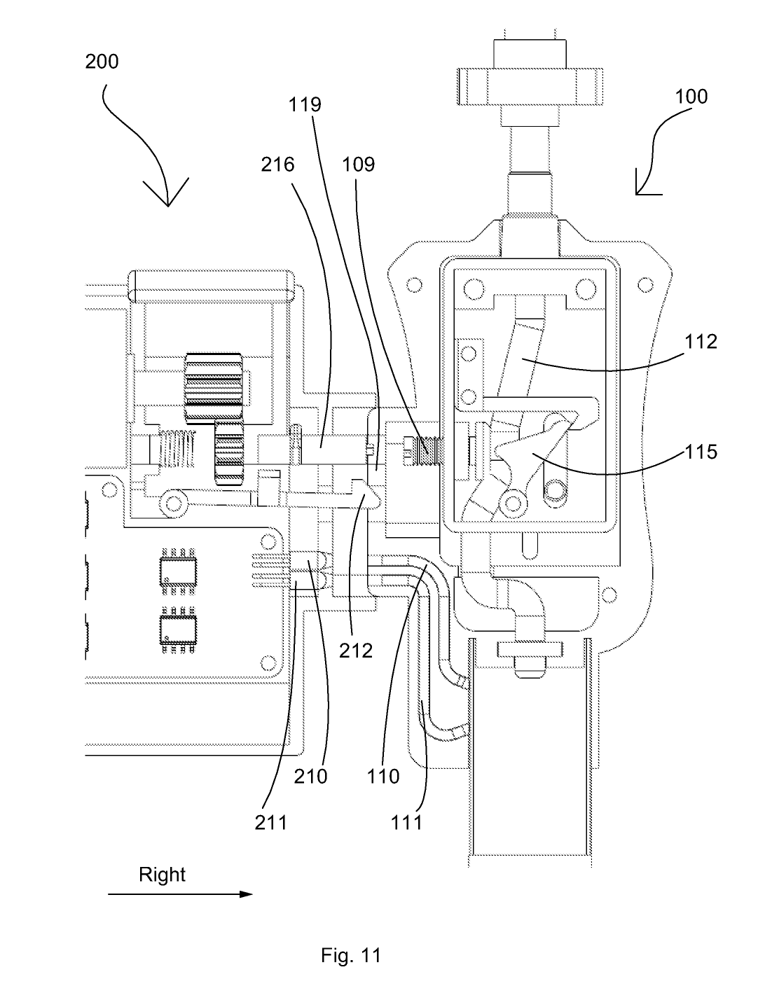

FIG. 11 is a schematic side view illustration of an exemplary embodiment of a smart valve 100, which is connected to a control unit 200, according to the present invention. The present illustration shows the state of the components of the smart valve 100 and the control unit 200, shown only in part, in the first stage of their connection process, while the control unit 200 moves right, as shown by the arrow in the illustration.

In this first stage the control unit lock 212 slides towards the locking wall 119 and the locking shaft 216 is in a state of “spring wound” toward the drop controller means 109.

The optical transmitter 210 is facing the transmitter light guide 110, and the optical receiver 211 is facing the receiver light guide 111.

The smart valve 100 is in closed mode, which prevents dripping or continuous flow through the internal tubule 112, by means of the lock 115.

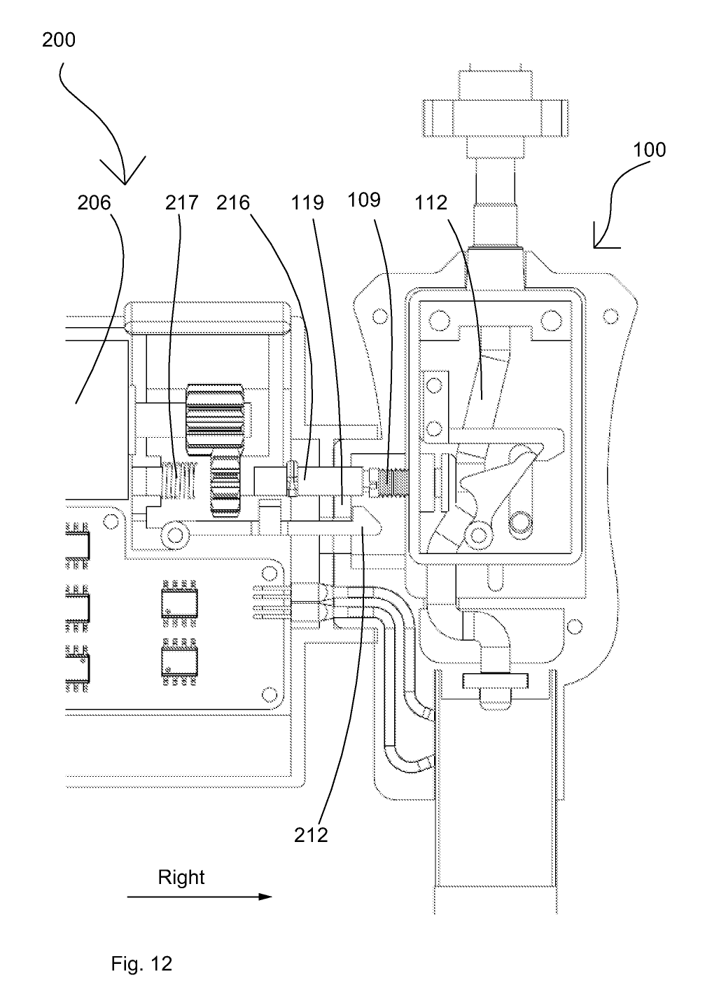

FIG. 12 is a schematic side view illustration of an exemplary embodiment of a smart valve 100, which is connected to a control unit 200, according to the present invention. This illustration shows the state of the components of the smart valve 100 and the control unit 200, which is shown in part, in the second stage of their connection process.

In this second stage, the control unit 200, with further movement to the right, in the direction of the arrow shown in the illustration, is locked to the smart valve 100. The control unit lock 212 goes through the locking wall 119 and is locked onto it. The locking motion of the lock 212 is an angular movement which can be generated by a spring, not shown in the illustration: while in this case, the lock 212 has freedom of angular movement around an axis near its left end, or by means of elasticity of the locking wall 119. In this case, it is harnessed at its left end, or with any other suitable device.

At this point, the engagement of the locking shaft 216 with the drop controller means 109 starts, similar to the engagement of a screwdriver with the head of a screw, while the locking shaft 216 is rotated by the step motor 206 and pressed to the right for the purpose of engagement by the spring 217 for no more than one full revolution until the engagement is complete. At the end of this second stage, passage of fluid through the internal tubule 112 is not possible.

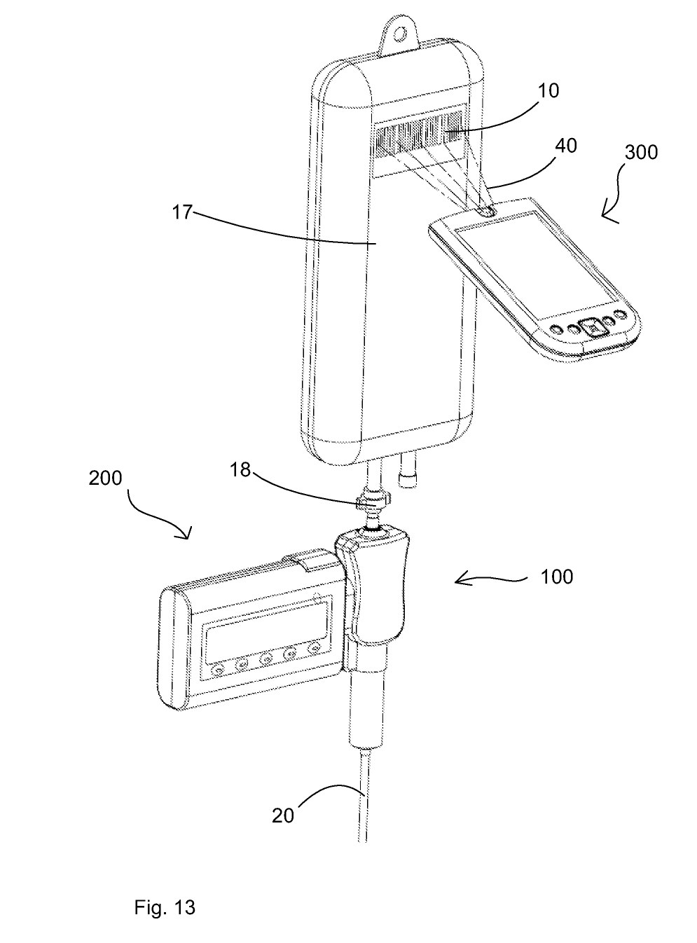

FIG. 13 is a schematic perspective view illustration of an exemplary embodiment of a smart valve 100, integrated with control unit 200 and connected between an IV bag 17 and the infusion tubule 20, according to the present invention.

The hand-held computer 300 scans the infusion bag barcode sticker 10, by means of IR radiation 40, or by means of any other suitable radiation such as RFID, and compares the code entered into hand-held computer 300 and the scanned code, which is entered into its memory.

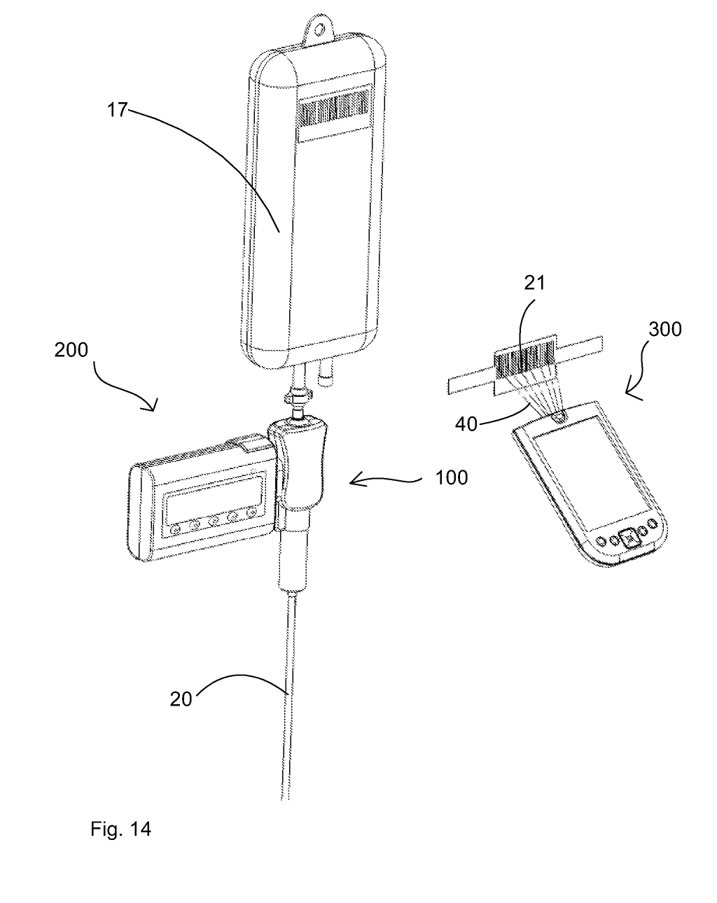

FIG. 14 is a schematic perspective view illustration of an exemplary embodiment of a smart valve 100, integrated with control unit 200 and connected between an IV bag 17 and the infusion tubule 20, according to the present invention.

The hand-held computer 300 scans the wristband patient barcode 21 by means of IR radiation 40, or any other suitable radiation such as RFID, and compares the code entered into it with the wristband patient barcode 21 which is scanned and entered into its memory.

FIG. 15 is a schematic perspective view illustration of an exemplary embodiment of a smart valve 100, integrated with the control unit 200 and connected between an IV bag 17 and the infusion tubule 20, according to the present invention.

After scanning the infusion bag barcode sticker 10 and the wristband patient barcode 21, duplex wireless communication 41 is established between the hand-held computer 300, and the control unit 200. If all of the data is authenticated, the hand-held computer 300 enables control unit 200 to continue as activated.

The duplex wireless communication 41 is maintained by a control unit wireless communication subsystem 220 and a hand-held computer wireless communication subsystem 304 which can be a little chip on a board of the hand-held computer 300.

The hand-held computer 300 is capable of transmitting all of the data, such as time, dosage, and quantity data, through the wireless communication 41.

During its entire process, the control unit 200 transmits data regarding the dripping rate and quantity at any given time. When the required dose is given, or according to any other criterion, the control unit 200 sends an end message to hand-held computer 300 and all of the data is registered in real time.

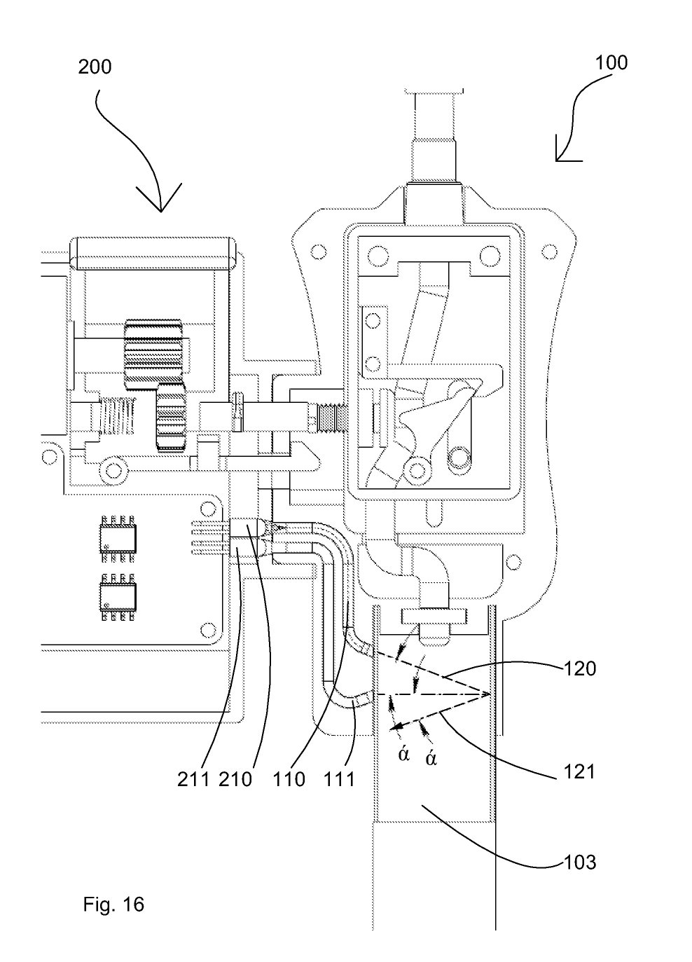

FIG. 16 is a schematic side view illustration of an exemplary embodiment of a smart valve 100, which is connected to a control unit 200, according to the present invention. This illustration shows the state of the components of the smart valve 100 and the control unit 200, shown only in part, at a stage in which they cannot be disconnected from each other, and a process of dripping sensing is started.

The optical transmitter 210 transmits its transmission signals as an AC light wave in order to prevent background light interference. The light waves pass through the transmitter light guide 110 and because there is no dripping, the amount of light that returns to the receiver light guide 111 is minimal and does not exceed the threshold necessary for recognizing a proper signal level.

The transmitted light ray 120 hits the wall of the dripping chamber 103.

The transmitted light ray 120 hits the wall at angle \acute{α} relative to the perpendicular to the wall and is reflected, as a reflected light ray 121, at angle \acute{α}, with the perpendicular serving as a symmetry line, all practically on the same plane.

Note: the light ray may be reflected from the wall, however the reflection is minimal due to the acute angle.

The reflected light ray 121 in the above described situation is not directed such that it can enter the receiver light guide 111, and thus provides a signal, which is minimally under threshold, for reception by the optical receiver 211.

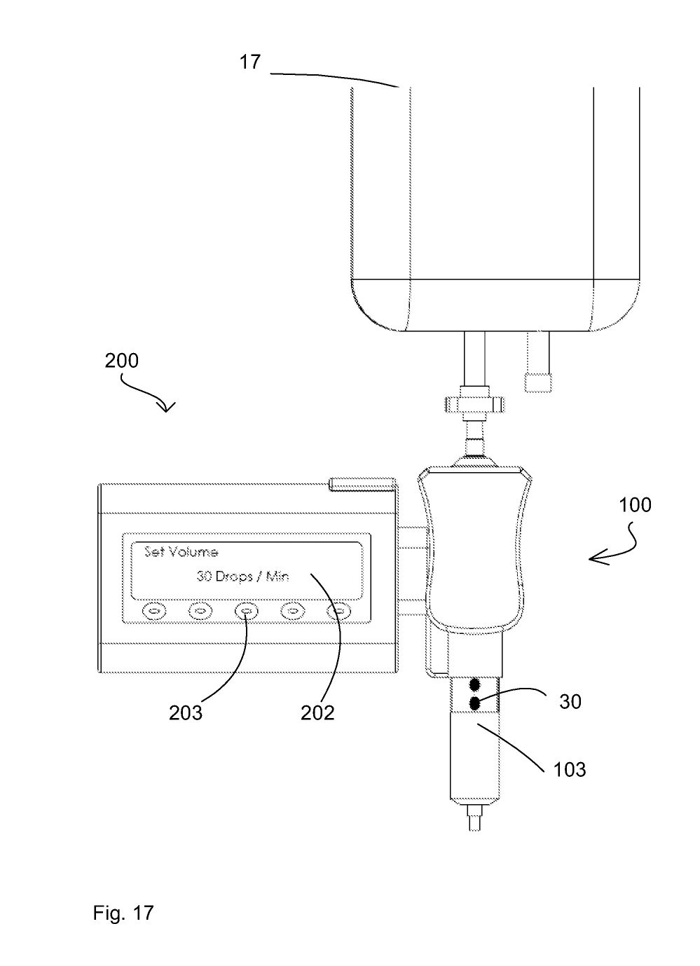

FIG. 17 is a schematic perspective view illustration of an exemplary embodiment of a smart valve 100, integrated with a control unit 200 and connected between an IV bag 17 and the infusion tubule 20, according to the present invention, during adjustment of the control unit 200. The adjustment is achieved by entering data into keyboard 203 and receiving results on display 202. After the control unit 200 activates the smart valve 100, the flow of fluid is enabled, and fluid drops 30 begin to appear in dripping chamber 103.

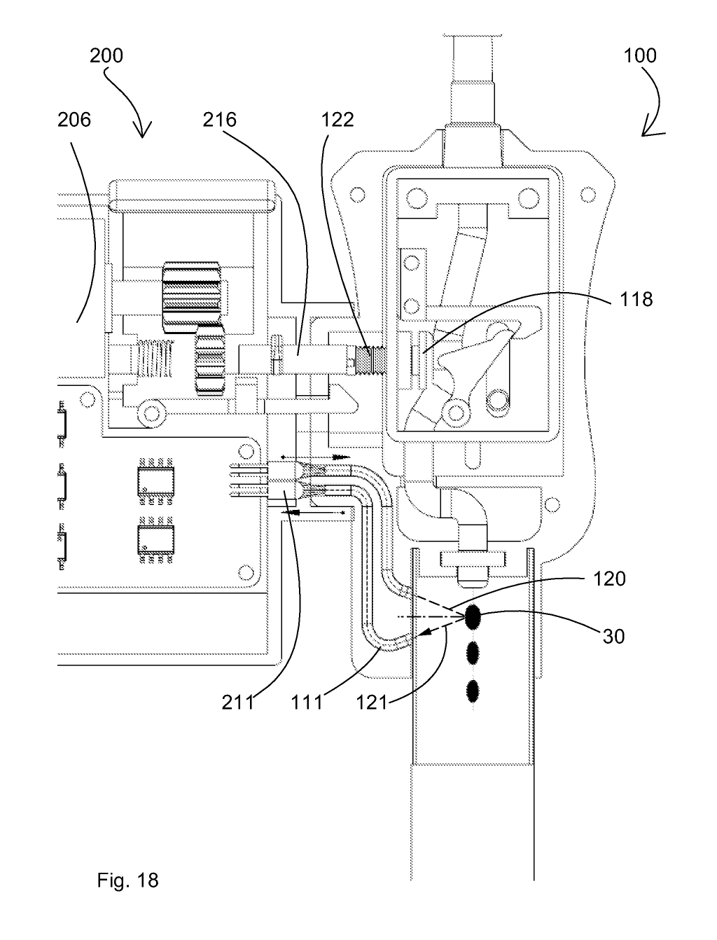

FIG. 18 is a schematic side view illustration of an exemplary embodiment of a smart valve 100, connected to a control unit 200, according to the present invention.

The present illustration shows the state of the components of the smart valve 100 and the control unit 200, shown only in part, at the stage in which the control unit 200 recognizes drops. The recognition of drops occurs when the course of the light, as described in FIG. 16, changes when a fluid drop 30, which goes through the transmitted light ray 120, is disposed in a suitable geometrical location. The fluid drop 30 reflects the light such that the reflected light ray 121 enters the receiver light guide 111, and is received through it in the optical receiver 211. The microcontroller 208 calculates the elapsed time between two consecutive fluid drops 30 and activates the step motor 206 for the purpose of opening or closing, if required, according to the data entered in the keyboard.

The microcontroller 208 uses closed loop control, and during the entire time of activation monitors the state of the step motor 206, which controls movement to the left and right (relative to the illustration plane) of the drop controller means plane 118.

This is achieved also by means of rotating the integral screw 122, which is an integral part of the locking axis 216. Closing the integral screw 122 will reduce the flow rate, which as noted is a dripping rate, while opening it will increase the rate.

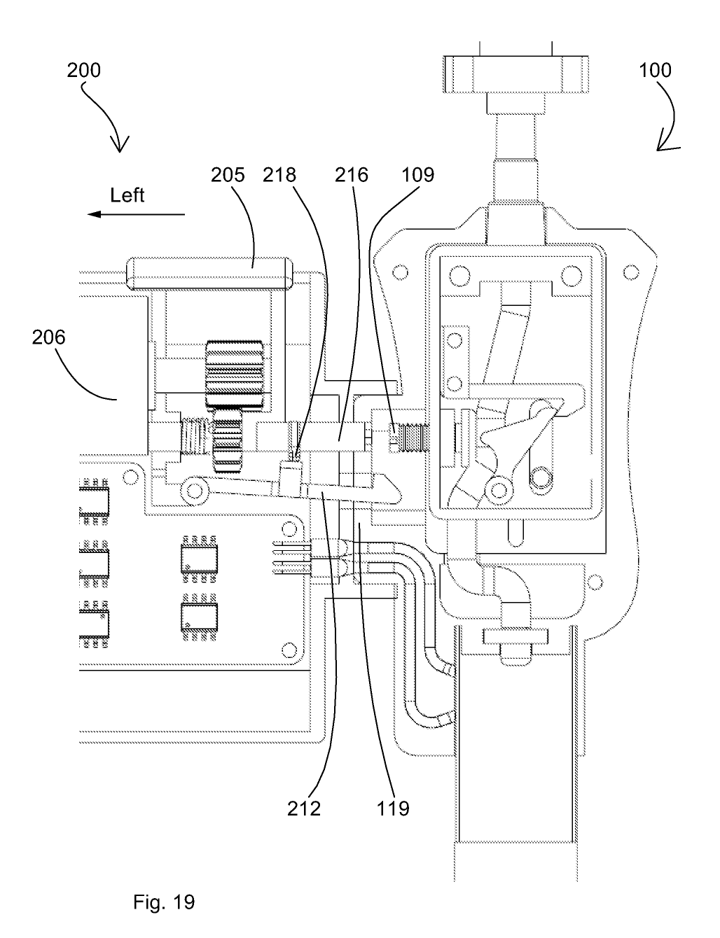

FIG. 19 is a schematic side view illustration of an exemplary embodiment of a smart valve 100, connected to a control unit 200, according to the present invention.

The present illustration shows the state of the components of the smart valve 100 and the control unit 200, shown only in part, at the stage in which the control unit 200 is constantly monitoring the dripping rate. As soon as the dripping stops, for any reason, for longer than a given time, such as 30 seconds, the control unit 200 closes the smart valve 100 hermetically, and the display 202 displays a message such as “The system can be disconnected”. Disconnection is performed by pulling switch 205 to the left, as shown by the arrow in the illustration, causing the step motor 206 to start rotating to opening position, the cam 218 is in the upper position in the end of the switch 205 position. The step motor 206 rotates by 180 degrees and the cam 218 pushes the control unit lock 212 down. The position of the switch 205 is monitored by cutoff detectors, not shown in the illustration, causing the release of the locking shaft 216 from the drop controller means 109 and the automatic activation of the step motor 206 to open state of the control unit lock 212. Opening the control unit lock 212 is performed by pressing cam 218, which is connected to locking shaft 216, towards the locking wall 119, enabling the disconnection of the control unit lock 212 from the smart valve 100, causing the release of locking shaft 216 from the drop controller means 109.

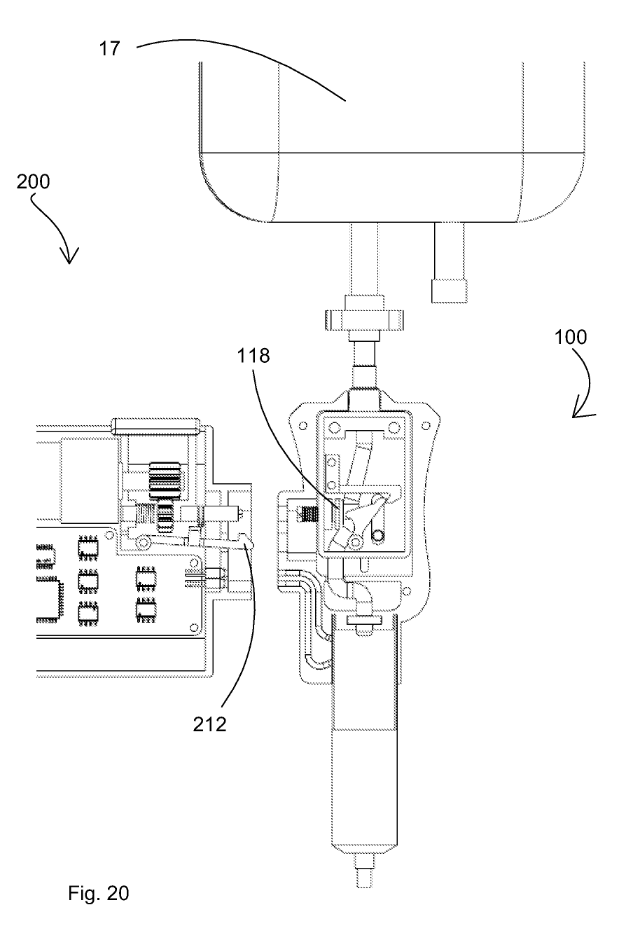

FIG. 20 is a schematic side view illustration of an exemplary embodiment of a smart valve 100, connected between an IV bag 17 and the infusion tubule 20, according to the present invention, in the stage following disconnection from the control unit 200.

The control unit 200 is in closed mode, the drop controller means plane 118, and the control unit lock 212 is in open mode and enables further activation (with another smart valve 100).

While the invention has been described with respect to a limited number of embodiments, it will be appreciated that many variations, modifications and other applications of the invention may be made, such as designing drop controlling and counting valve on key system 1000 in various configurations, for example in order to obtain the desired position of the center of gravity by changing the positions of various components and even adding balancing weights.

What is claimed is:

1. A drop controlling and counting valve on key system for ensuring authentication and for controlling the rate of flow of medications, in liquid state drops, under control of an authentication unit, the authentication unit containing characteristics of the medication fluid and details of the patient, for calculating a correlation value between the details and the characteristics, the drop controlling and counting valve on key system comprising:
(A) a smart valve including:
(i) an immovable assembly including:
(a) a smart valve to control unit connector; and
(B) a control unit including:
(i) a control unit to smart valve connector, wherein said smart valve to control unit connector and said control unit to smart valve connector are compatible; and
(ii) a control unit wireless communication subsystem.

2. The drop controlling and counting valve on key system of claim 1 further comprising:
(C) a hand-held computer including:
(i) a hand-held computer wireless communication subsystem, wherein said control unit wireless communication subsystem and said hand-held computer wireless communication subsystem are compatible.

3. The drop controlling and counting valve on key system of claim 1 wherein the immovable assembly further includes:
(b) a lock pin, having no movement capability relative to said immovable assembly;
(c) a dripping chamber positioned at a lower section of said immovable assembly at times of a normal operation;
(d) a lower connector attached to said dripping chamber;
(e) a transmitter light guide disposed between said dripping chamber and said smart valve to control unit connector; and
(f) a receiver light guide disposed between said dripping chamber and said smart valve to control unit connector.

4. The drop controlling and counting valve on key system of claim 3 wherein the smart valve further includes:
(ii) a moveable assembly, wherein said moveable assembly has a limited movement capability within said immovable assembly, and wherein said immovable assembly includes:
(a) a spike having a shape and dimensions suitable for insertion in a IV bag first port.

5. The drop controlling and counting valve on key system of claim 4 wherein the smart valve further includes:
(iii) a internal tubule disposed between said spike and said lower connector.

6. The drop controlling and counting valve on key system of claim 5 wherein the moveable assembly further includes:
(b) a lock having angular movement capability, wherein said lock does not block flow of fluid within said internal tubule during times of storage;
(c) a lock hook for locking said lock in a position pressing on said internal tubule; and
(d) a drop controller means for controlling the rate of fluid dripping through said internal tubule.

7. The drop controlling and counting valve on key system of claim 3 wherein the control unit further includes:
(iii) an optical transmitter, wherein when said control unit is engaged to said smart valve, said optical transmitter is positioned opposite said transmitter light guide;
(iv) an optical receiver, wherein when said control unit is engaged to said smart valve, said optical transmitter is positioned opposite said receiver light guide; and
(v) a control unit locker having angular movement capability, and wherein when said control unit is connected to said smart valve, said control unit locker can prevent disengagement of said control unit from said smart valve.

8. The drop controlling and counting valve on key system of claim 7 wherein the control unit further includes:
(vi) a locking shaft having rotational movement capability;
(vii) a combining ligule disposed as part of said locking shaft, wherein said combining ligule has shape and dimensions suitable for engagement with said drop controller means; and
(viii) a cam disposed as part of said locking shaft, wherein said cam has a shape and dimensions suitable for moving said control unit lock in order to enable disengagement of said control unit from said smart valve.

9. The drop counting valve on key system of claim 8 wherein the control unit further includes:
(ix) a step motor, said step motor having a step motor shaft;
(x) a first cogwheel disposed at said step motor shaft; and
(xi) a second cogwheel disposed at said locking shaft, wherein said first cogwheel and said second cogwheel constitute a control transmission.

10. The drop controlling and counting valve on key system of claim 9 wherein the control unit further includes:
(xii) a microcontroller having the capability of operating said step motor; and
(xiii) a power source, for supplying power to said step motor and to said microcontroller.

11. The drop controlling and counting valve on key system of claim 3 wherein the control unit further includes:
(iii) an optical transmitter, wherein when said control unit is engaged to said smart valve, said optical transmitter is positioned opposite said transmitter light guide;
(iv) an optical receiver, wherein when said control unit is engaged to said smart valve, said optical transmitter is positioned opposite said receiver light guide;
(v) a control unit lock having angular movement capability, and wherein when said control unit is connected to said smart valve, said control unit lock can prevent disengagement of said control unit from said smart valve;
(vi) a locking shaft having rotational movement capability;
(vii) a combining ligule disposed as part of said locking shaft, wherein said combining ligule has a shape and dimensions suitable for engagement with said drop controller means;
(viii) a cam disposed as part of said locking shaft, wherein said cam has a shape and dimensions suitable for moving said control unit lock in order to enable disengagement of said control unit from said smart valve;
(ix) a step motor, said step motor having a step motor shaft;
(x) a first cogwheel disposed at said step motor shaft;
(xi) a second cogwheel disposed at said locking shaft, wherein said first cogwheel and said second cogwheel constitute a control transmission;
(xii) a microcontroller having the capability of operating said step motor; and
(xiii) a power source, for supplying power to said step motor and to said microcontroller.

12. A method for ensuring authentication and for controlling the rate of flow of medications, in liquid state drops, infused in IV bags, the method comprising the stages of:
(A) providing a drop controlling and counting valve on key system, said drop controlling and counting valve on key system including:
(i) a first smart valve having a spike;
(ii) a control unit; and
(iii) a hand-held computer;
(B) inserting said spike in an IV bag port, wherein said insertion causes a state of prevention of fluid flow from said IV bag through said first smart valve;
(C) connecting said control unit to said first smart valve;
(D) scanning a vial barcode sticker and a wristband patient barcode by said hand-held computer, and assessing an authentication;
(E) opening a pass which enables flow of fluid through said first smart valve; and
(F) measuring the flow rate of fluid, by counting fluid drops passing through said first smart valve, over a given period of time, wherein the average mass of a drop is known.

13. The method for ensuring authentication and for controlling the rate of flow of medications, in liquid state drops, infused in IV bags, of claim 12 further comprising the stages of:
(G) calculating an amount of fluid mass passing through said first smart valve; and
(H) preventing flow of fluid through said first smart valve, after finding that a fluid mass of a predetermined amount passed through said first smart valve.

14. The method for ensuring authentication and for controlling the rate of flow of medications, in liquid state drops, infused in IV bags, of claim 13 further comprising the stages of:
(I) disconnecting said control unit from said first smart valve; and
(J) extracting said spike from said IV bag port.

15. The method for ensuring authentication and for controlling the rate of flow of medications, in liquid state drops, infused in IV bags, of claim 14 further comprising the stages of:
(K) destroying said first smart valve.

16. The method for ensuring authentication and for controlling the rate of flow of medications, in liquid state drops, infused in IV bags, of claim 14 further comprising the stages of:
(L) inserting a spike of a second smart valve in an IV bag port, wherein said insertion causes a state of prevention of fluid flow from said IV bag through said second smart valve; and
(M) connecting said control unit to said second smart valve.
